(19) United States
(12) Patent Application Publication
(10) Pub. No.: US 2026/0090861 A1
(43) Pub. Date: Apr. 2, 2026

(54) MAGNETICALLY COUPLABLE FIDUCIAL MARKER ARRAYS

(71) Applicant: Medivis, Inc., New York, NY (US)

(72) Inventors: Jesse Harrison Gallant, New York, NY (US); Osamah Choudhry, New York, NY (US); Christopher Morley, New York, NY (US); Florentin Jonas Liebmann, New York, NY (US)

(21) Appl. No.: 19/344,170

(22) Filed: Sep. 29, 2025

Related U.S. Application Data
(60) Provisional application No. 63/700,747, filed on Sep. 29, 2024.
(60) Provisional application No. 63/700,748, filed on Sep. 29, 2024.

(51) Int. Cl. A61B 90/00; A61B 17/00

(52) U.S. Cl. CPC A61B 90/39; A61B 17/00; A61B 2017/00477; A61B 2017/00876; A61B 2090/3916; A61B 2090/3991

(57) ABSTRACT

A magnetically couplable fiducial marker array can include a rigid body, a plurality of surgical fiducial markers, and a magnetic coupling arrangement. The rigid body can be configured to support the plurality of surgical fiducial markers at fixed positions relative to each other along the rigid body to form a fixed positional arrangement of surgical fiducial markers. At least a support portion of the rigid body can be arranged along a surgical fiducial marker support plane and the fixed positional arrangement of surgical fiducial markers can be asymmetrical.

The magnetic coupling arrangement can be formed at the rigid body with array magnetic components and can be configured to form a magnetic coupling between the array magnetic components and separate magnetic components of a separate surgical spinous process clamp at a first fixed position relative to the separate surgical spinous process clamp.

### CROSS REFERENCE TO RELATED APPLICATIONS

This application claims the benefit of priority to U.S. Provisional Application Nos. 63/700,747, filed Sep. 29, 2024, and 63/700,748, filed Sep. 29, 2024, which are hereby incorporated by reference in their entirety. This application is also related to commonly owned U.S. patent application Ser. Nos. 18/135,663, filed Apr. 17, 2023, Ser. No. 18/135,674, filed Apr. 17, 2023, and Ser. No. 18/900,106, filed Sep. 27, 2024, all applications of which are hereby incorporated by reference in their entireties herein.

### TECHNICAL FIELD

The present disclosure relates generally to medical devices, and more particularly to surgical tools and devices used during medical surgeries and procedures.

### BACKGROUND

Planning and navigation are necessary for many medical procedures, and surgical teams typically have a plan based on medical imagery before ever entering an operating room. Conventional medical imaging systems such as X-ray, MRI, CT, and others have limitations regarding two-dimensional and three-dimensional images, however, and surgeons often need to consider numerous image views and slices to plan surgical procedures. Recent medical advances leverage these applications of medical imagery and surgical plans by using a computer-aided augmented reality environment, which can allow for the tracking of patients and physical instruments during surgical procedures by using fiducial markers and tracking components.

Unfortunately, conventional tracking systems are often limited in their ability to accurately generate, render, and apply virtual interactions in an augmented reality environment based on the orientations and positions of physical instruments with respect to those of physical landmarks identified on a patient body, particularly when things move during surgery. Unstable or unreliable positioning of fiducial markers can play a role in these issues. Limited or inaccurate tracking can then affect the overall performance of such systems during surgical procedures, and the need for accuracy in this regard can lead to overly cumbersome or complex attachment devices and systems.

While traditional ways of virtually tracking items during surgery have worked well in the past, improvements are always helpful. In particular, what is desired are medical systems and devices that facilitate the stable and reliable positioning of fiducial markers during surgery in a simple and streamlined manner.

### SUMMARY

It is an advantage of the present disclosure to provide medical systems and devices that facilitate the stable and reliable positioning of fiducial markers during surgery in a simple and streamlined manner. The disclosed features, apparatuses, systems, and methods relate to fiducial marker arrays that can be used to locate fiducial markers and other devices during surgeries and other medical procedures. In particular, the disclosed systems and methods can involve magnetically couplable fiducial marker arrays that can reliably locate fiducial markers relative to a patient and that can be reliably coupled to one or more other surgical devices or items, such as a surgical spinous process clamp.

In various embodiments of the present disclosure, a fiducial marker array can include at least a rigid body and a coupling arrangement coupled to the rigid body. The rigid body can be configured to support a plurality of surgical fiducial markers at fixed positions relative to each other along the rigid body to form a fixed positional arrangement of surgical fiducial markers.

At least a support portion of the rigid body can be arranged along a surgical fiducial marker support plane and the fixed positional arrangement of surgical fiducial markers can be asymmetrical. The coupling arrangement can be configured to couple the fiducial marker array to a separate surgical device at a first fixed position relative to the separate surgical device.

In various detailed embodiments, the fiducial marker array can also include the plurality of surgical fiducial markers. The separate surgical device can be a surgical spinous process clamp. In some arrangements, the coupling arrangement can include one or more array magnetic components configured to form a magnetic coupling to one or more separate magnetic components of the separate surgical device. The magnetic coupling can result in the fixed positional arrangement of surgical fiducial markers being set at a constant orientation and location with respect to the separate surgical device while the fiducial marker array remains magnetically coupled to the separate surgical device. The constant orientation and location can be repeatable when the fiducial marker array is uncoupled from and recoupled to the separate surgical device such that recalibration of the fiducial marker array is unnecessary. The support portion of the rigid body can be configured to support all of the plurality of surgical fiducial markers. Also, the support portion of the rigid body can include a primary branch and multiple secondary branches extending from the primary branch at different locations. Each secondary branch can include at least one location configured to support one of the plurality of surgical fiducial markers. The multiple secondary branches can include at least four secondary branches. In some arrangements, the rigid body can extend laterally in only one direction away from the coupling arrangement. In other arrangements, the rigid body can extend laterally in exactly two opposite directions away from the coupling arrangement.

In further embodiments of the present disclosure, a magnetically couplable fiducial marker array can include a rigid body, a plurality of surgical fiducial markers, and a magnetic coupling arrangement. The rigid body can be configured to support the plurality of surgical fiducial markers at fixed positions relative to each other along the rigid body to form a fixed positional arrangement of surgical fiducial markers. At least a support portion of the rigid body can be arranged along a surgical fiducial marker support plane and the fixed positional arrangement of surgical fiducial markers can be asymmetrical. The magnetic coupling arrangement can be formed at the rigid body and can have one or more array magnetic components. The magnetic coupling arrangement can be configured to form a magnetic coupling between the one or more array magnetic components and one or more separate magnetic components of a separate surgical spinous process clamp at a first fixed position relative to the separate surgical spinous process clamp.

In still further embodiments of the present disclosure, various methods of using a magnetically couplable fiducial marker array are provided. Pertinent process steps can include removably coupling a plurality of surgical fiducial markers to a magnetically couplable full fiducial marker array at fixed positions relative to each other to form an asymmetrical fixed positional arrangement of surgical fiducial markers, forming a magnetic coupling between the magnetically couplable full fiducial marker array and a separate surgical component, and calibrating the locations of the plurality of surgical fiducial markers while the magnetically couplable full fiducial marker array is magnetically coupled to the separate surgical component. Additional process steps can include removing the magnetically couplable full fiducial marker array from the separate surgical component, forming a magnetic coupling between a magnetically couplable half fiducial marker array and the separate surgical component, and reading automatically surgical fiducial markers on the magnetically couplable half fiducial marker array by an augmented reality system while the magnetically couplable half fiducial marker array is magnetically coupled to the separate surgical component. The separate surgical component can be a surgical spinous process clamp. Other apparatuses, methods, features, and advantages of the disclosure will be or will become apparent to one with skill in the art upon examination of the following figures and detailed description. It is intended that all such additional apparatuses, methods, features and advantages be included within this description, be within the scope of the disclosure, and be protected by the accompanying claims.

### BRIEF DESCRIPTION OF THE DRAWINGS

The included drawings are for illustrative purposes and serve only to provide examples of possible structures, arrangements, and methods of use for magnetically couplable fiducial marker arrays. These drawings in no way limit any changes in form and detail that may be made to the disclosure by one skilled in the art without departing from the spirit and scope of the disclosure.

FIG. 1A illustrates in front perspective view an example surgical fiducial marker system having a surgical fiducial marker positioner and spinous process clamp coupled to a patient spine according to one embodiment of the present disclosure.

FIG. 1B illustrates in side perspective view the surgical fiducial marker system of FIG. 1A coupled to the patient spine according to one embodiment of the present disclosure.

FIG. 2A illustrates in front perspective view an example surgical fiducial marker system having a surgical fiducial marker positioner rotationally coupled to a spinous process clamp according to one embodiment of the present disclosure.

FIG. 2B illustrates in top plan view the surgical fiducial marker system of FIG. 2A with the surgical fiducial marker positioner oriented perpendicular to the spinous process clamp according to one embodiment of the present disclosure.

FIG. 3A illustrates in side perspective view an example surgical fiducial marker positioner at a first fixed rotational position relative to a coupled spinous process clamp according to one embodiment of the present disclosure.

FIG. 3B illustrates in side perspective view the surgical fiducial marker positioner of FIG. 3A at a second fixed rotational position relative to the coupled spinous process clamp according to one embodiment of the present disclosure.

FIG. 4 illustrates a flowchart of an example summary method of using a surgical fiducial marker positioner according to one embodiment of the present disclosure.

FIG. 5A illustrates in top perspective view an example arm segment of a surgical fiducial marker positioner according to one embodiment of the present disclosure.

FIG. 5B illustrates in side cross section view the arm segment of FIG. 5A according to one embodiment of the present disclosure.

FIG. 5C illustrates in side perspective view an example alternative surgical fiducial marker positioner arm segment with a fiducial marker and fiducial marker coupler coupled thereto according to one embodiment of the present disclosure.

FIG. 6A illustrates in top plan view an example alternative surgical fiducial marker positioner according to one embodiment of the present disclosure.

FIG. 6B illustrates in top plan view an example further alternative surgical fiducial marker positioner according to one embodiment of the present disclosure.

FIG. 7A illustrates in perspective view an example positioner coupling component for a surgical fiducial marker positioner according to one embodiment of the present disclosure.

FIG. 7B illustrates in perspective view the positioner coupling component of FIG. 7A rotationally coupled to a clamp coupling component for a spinous process clamp according to one embodiment of the present disclosure.

FIG. 8A illustrates in perspective view an example alternative positioner coupling component for a surgical fiducial marker positioner according to one embodiment of the present disclosure.

FIG. 8B illustrates in perspective view the alternative positioner coupling component of FIG. 8A rotationally coupled to a clamp coupling component for a spinous process clamp according to one embodiment of the present disclosure.

FIG. 9 illustrates a flowchart of an example detailed method of using a surgical fiducial marker positioner according to one embodiment of the present disclosure.

FIG. 10A illustrates in front perspective view an example alternative surgical fiducial marker system having a surgical spinous process clamp and a magnetically couplable fiducial marker array according to one embodiment of the present disclosure.

FIG. 10B illustrates in top plan view the alternative surgical fiducial marker system of FIG. 10A according to one embodiment of the present disclosure.

FIG. 11 illustrates in front perspective view an example magnetic coupling between a surgical spinous process clamp and a magnetically couplable fiducial marker array according to one embodiment of the present disclosure.

FIG. 12A illustrates in front perspective view the magnetic coupling of FIG. 11 being undone by removing the magnetically couplable fiducial marker array from the surgical spinous process clamp according to one embodiment of the present disclosure.

FIG. 12B illustrates in front perspective view the magnetically couplable fiducial marker array being completely removed from the surgical spinous process clamp of FIG. 12A according to one embodiment of the present disclosure.

FIG. 13A illustrates in side cross-section view the magnetic coupling between a surgical spinous process clamp and a magnetically couplable fiducial marker array of FIG. 11 according to one embodiment of the present disclosure.

FIG. 13B illustrates in perspective cross-section view the magnetic coupling of FIG. 13A according to one embodiment of the present disclosure.

FIG. 14A illustrates in top perspective view another example alternative surgical fiducial marker system having a surgical spinous process clamp and a magnetically couplable half fiducial marker array according to one embodiment of the present disclosure.

FIG. 14B illustrates in bottom perspective view the alternative surgical fiducial marker system of FIG. 14A according to one embodiment of the present disclosure.

FIG. 15 illustrates a flowchart of an example method of using a magnetically couplable fiducial marker array according to one embodiment of the present disclosure.

FIG. 16 illustrates in top perspective view yet another alternative surgical fiducial marker system with the spinous process clamp of FIG. 2A and an alternative surgical fiducial marker array according to one embodiment of the present disclosure.

### DETAILED DESCRIPTION

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

As is generally well known, modern surgical plans and procedures are sometimes facilitated by using a computer-aided augmented reality environment. Surgical fiducial markers can be used for tracking of patients and physical instruments during surgical procedures in overall systems that can also include specialized lighting arrangements, cameras, and computing systems. Attachment devices are often used to affix the surgical fiducial markers in place relative to the patient, and unstable or unreliable positioning of these markers can result in the reduced effectiveness of the overall computer-aided augmented reality environment. The disclosed surgical attachment devices are specifically designed to facilitate the stable and reliable positioning of fiducial markers during surgery in a simple and streamlined manner.

The present disclosure relates in various embodiments to features, apparatuses, systems, and methods of use for surgical positioning devices, and in particular surgical fiducial marker positioners. This can generally involve positioners or other suitable locating devices that are configured to facilitate the stable and reliable positioning of fiducial markers during surgery in a simple and streamlined manner. In specific arrangements, this can involve surgical fiducial marker positioners that can reliably locate fiducial markers relative to a patient and that can be coupled to one or more other surgical devices or items, such as a spinous process clamp.

In various embodiments of the present disclosure, novel surgical fiducial marker positioners can include at least a rigid frame or other suitable structure and a positioner coupling component coupled thereto. The rigid frame can support a plurality of surgical fiducial markers at fixed positions relative to each other along the rigid frame to form a fixed positional arrangement of surgical fiducial markers that is asymmetrical. The surgical fiducial markers and/or marker couplers that removably couple the surgical fiducial markers to the frame can also be part of a surgical fiducial marker positioner. The positioner coupling component can couple the surgical fiducial marker positioner to a separate surgical component at a first fixed position relative to the separate surgical component, which can be a spinous process clamp.

Although various embodiments disclosed herein discuss the use of a surgical fiducial marker positioner in conjunction with a spinous process clamp as part of an overall surgical fiducial marker system, it will be readily appreciated that the disclosed features, apparatuses, systems, and methods can also be used in conjunction with other devices and equipment that can leverage the advantages of a fiducial marker positioner. While the disclosed surgical fiducial marker positioners are contemplated for uses involving coupling to spinous process clamps that then clamp to spinous processes during a surgical procedure, it is specifically contemplated that other coupled objects and clamped targets and applications may also be applied. For example, the disclosed surgical fiducial marker positioners can couple to other body parts or other fixed items for surgical and non-surgical uses, such as during examination and testing procedures. Other devices and equipment that can be coupled to the disclosed surgical fiducial marker positioners can include alternative clamps and fixation components, which can clamp or affix the disclosed positioners to other body parts, lighting arrangements, sensing devices, or other desired equipment. Other applications, arrangements, and extrapolations beyond the illustrated embodiments are also contemplated.

The present disclosure also relates in various embodiments to features, apparatuses, systems, and methods of use for surgical positioning devices that are magnetically couplable fiducial marker arrays. Specifically, such fiducial marker arrays can include at least a rigid body and a coupling arrangement coupled to the rigid body. The rigid body can be configured to support a plurality of surgical fiducial markers at fixed positions relative to each other along the rigid body to form a fixed positional arrangement of surgical fiducial markers, and at least a support portion of the rigid body can be arranged along a surgical fiducial marker support plane with the fixed positional arrangement of surgical fiducial markers being asymmetrical. The coupling arrangement can be configured to couple the fiducial marker array to a separate surgical device at a first fixed position relative to the separate surgical device, which can be a surgical spinous process clamp. The coupling arrangement can include one or more array magnetic components configured to form a magnetic coupling to one or more separate magnetic components of the surgical spinous process clamp or other separate surgical device.

Although various embodiments disclosed herein discuss the use of a magnetically couplable fiducial marker array in conjunction with a surgical spinous process clamp as part of an overall surgical fiducial marker system, it will be readily appreciated that the disclosed features, apparatuses, systems, and methods can also be used in conjunction with other devices and equipment that can leverage the advantages of the disclosed devices and systems. While the disclosed magnetically couplable fiducial marker arrays are contemplated for uses involving coupling to surgical spinous process clamps that then clamp to spinous processes during a surgical procedure, it is specifically contemplated that other coupled objects and clamped targets and applications may also be applied. Other devices and equipment that can be coupled to the disclosed magnetically couplable fiducial marker arrays can include alternative clamps and fixation components, which can clamp or affix the disclosed arrays to other body parts, lighting arrangements, sensing devices, or other desired equipment. Other applications, arrangements, and extrapolations beyond the illustrated embodiments are also contemplated.

### Surgical Fiducial Marker Positioners

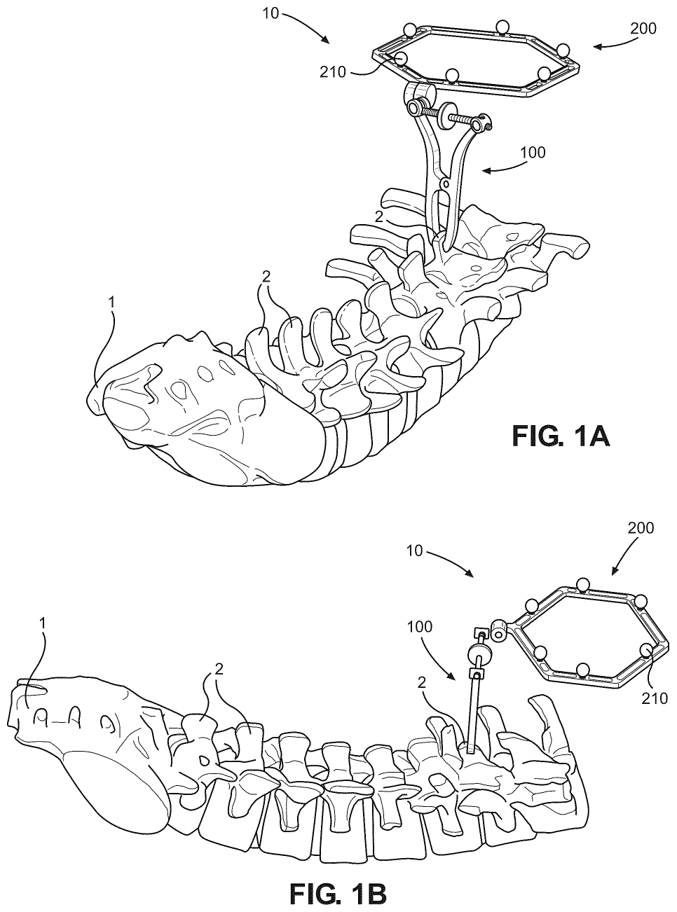

Referring first to FIGS. 1A and 1B, an example surgical fiducial marker system having a surgical fiducial marker positioner and spinous process clamp coupled to a patient spine is illustrated in front perspective and side perspective views respectively. Surgical fiducial marker system 10 can generally include a spinous process clamp 100 and a surgical fiducial marker positioner 200. Spinous process clamp 100 can be clamped to one of various spinous processes 2 along the spine 1 of a patient. Surgical fiducial marker positioner 200 can be coupled to spinous process clamp 100 and can be configured to position multiple surgical fiducial markers 210 with respect to the patent spine 1. As shown, spinous process clamp 100 can be firmly clamped to a spinous process and set at a fixed position relative to the patient and surgical fiducial marker positioner 200 can be firmly coupled to the spinous process clamp and set at a fixed position relative thereto, such that the surgical fiducial markers 210 are set at a fixed arrangement relative to the patient.

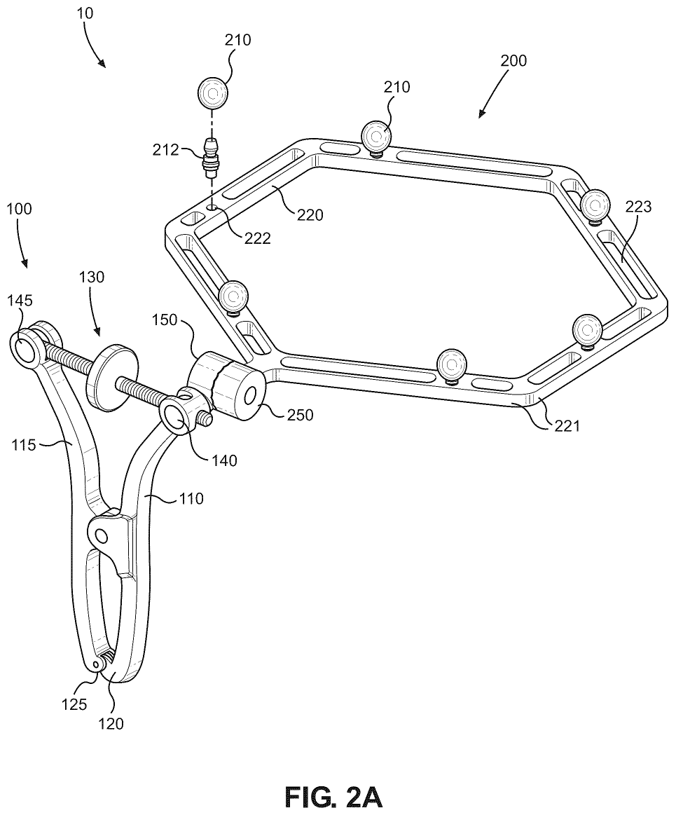

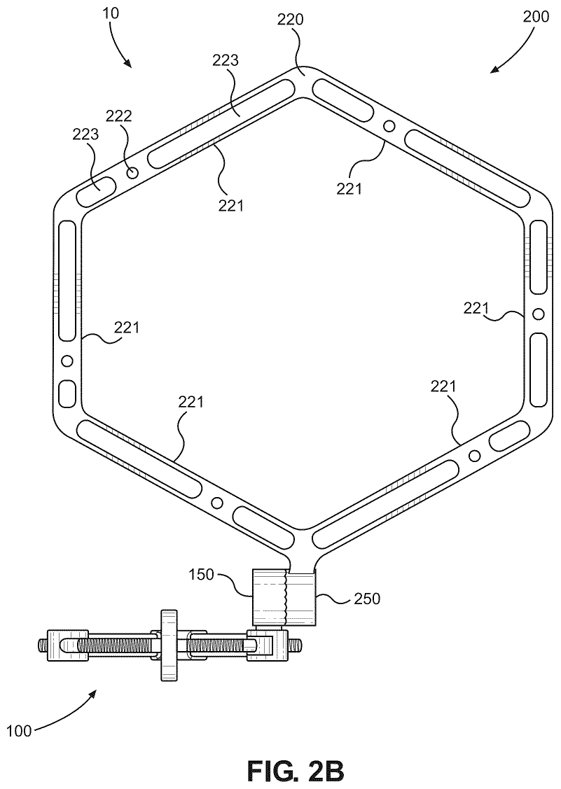

Continuing with FIGS. 2A and 2B, an example surgical fiducial marker system having a surgical fiducial marker positioner rotationally coupled to and oriented perpendicular to a spinous process clamp is shown in front perspective and top plan views respectively. Again, surgical fiducial marker system 10 can generally include a spinous process clamp 100 and a surgical fiducial marker positioner 200. Surgical fiducial marker positioner 200 can be coupled to spinous process clamp 100 by way of positioner coupling component 250 that can be configured to interact with clamp coupling component 150 of the clamp, as detailed below. In some embodiments, surgical fiducial marker positioner 200 can include surgical fiducial markers 210 coupled to a rigid frame 220 of multiple arm segments 221 by way of fiducial marker couplers 212 that insert into marker openings 222 in an asymmetrical fixed positional arrangement, among various other components and features as set forth in greater detail below.

In various embodiments, spinous process clamp 100 can include a first arm 110, a second arm 115, a first gripping component 120, a second gripping component 125, an adjustable spacing arrangement 130 including a first receiving bearing 140 and a second receiving bearing 145, and a clamp coupling component 150, among other possible components and features. While clamp coupling component 150 is shown as being located proximate the top of first arm 110, it will be readily appreciated that this coupling component can alternatively be located at various other places on spinous process clamp 100, such as proximate the top of second arm 115 for example. Further details regarding spinous process clamp 100 and variations thereof can be found in related U.S. patent application Ser. No. 18/135,663, titled “SPINOUS PROCESS CLAMP,” which application is again hereby incorporated by reference in its entirety.

As shown in FIG. 2B, the surgical fiducial markers and fiducial marker couplers can be removed from marker openings 222 in rigid frame 220, such that surgical fiducial marker positioner 200 can be a standalone device without these items in some arrangements. The same or new surgical fiducial markers and fiducial marker couplers can then be removably coupled to rigid frame 220. In various embodiments, surgical fiducial markers 210 can include infrared reflective spheres or balls, other retroreflective spheres, infrared-emitting diodes, or other items suitable for use with surgical optical tracking systems. Each surgical fiducial marker 210 can be coupled to an arm segment 221 along rigid frame 220 of surgical fiducial marker positioner 200 using a threaded coupler 212 that can be inserted into an opening of the fiducial marker as well as a marker opening 222 along the arm segment 221. Some or all of multiple arm segments 221 can have one or more marker openings 222 as well as one or more structural openings 223 therethrough, such as where it is desirable for the overall surgical fiducial marker positioner 200 to be lightweight and the remaining material has sufficient structural strength. All arm segments 221 and positioner coupling component 250 can be integrally formed into a single unit that can be comprised of medical grade plastic and/or stainless steel, among other possible arrangements and suitable materials.

The multiple arm segments 221 of surgical fiducial marker positioner 200 can be arranged into a rigid frame 220 arranged along a plane and having a polygon shape, such as six arm segments combining to form a hexagon, for example. Such a polygon shape can be irregular in some arrangements or may be symmetrical. The locations of marker openings 222 can form an asymmetrical pattern such that installation of surgical fiducial markers into all of the marker openings then results in a fixed positional arrangement of surgical fiducial markers that is asymmetrical such that it that will not cause ambiguity or confusion with the optical tracking system reading the locations of the fiducial markers. Although each arm segment 221 is shown as having a single marker opening 222 in FIG. 2B, it will be readily appreciated that each arm segment can have zero or multiple marker openings. In any such arrangement, it is contemplated that installing surgical fiducial markers into all or any subset of all marker openings 222 can still result in a fixed positional arrangement of surgical fiducial markers that is still asymmetrical.

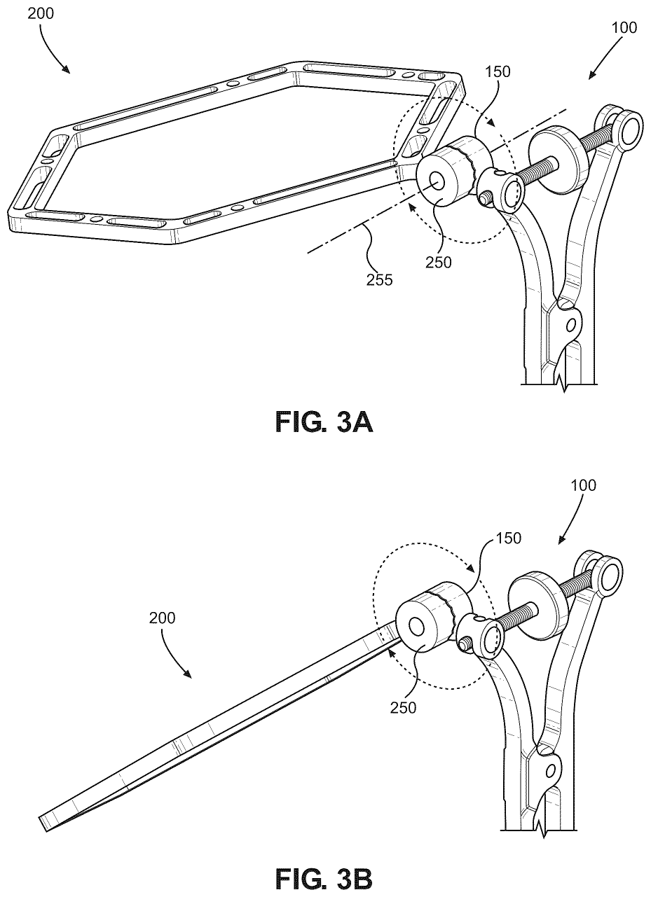

Focusing now on FIGS. 3A and 3B, an example surgical fiducial marker positioner is shown in side perspective views at first and second fixed rotational positions relative to a coupled spinous process clamp. In various arrangements, positioner coupling component 250 can be rotationally coupled to and configured to rotate with respect to clamp coupling component 150, which can be affixed to and stationary with respect to spinous process clamp 100. Rotation can be about a horizontal axis 255 that extends through the centers of positioner coupling component 250 and clamp coupling component 150, such that the position of entire surgical fiducial marker positioner 200 can be rotated about this axis. Changeable and different rotational positions may be desirable for a variety of reasons, such as to provide better visibility or usage of surgical fiducial markers to a separate surgical optical tracking system and/or to facilitate the structural stability and balance of overall surgical fiducial marker system 10, for example.

While the rotational orientation of surgical fiducial marker positioner 200 relative to spinous process clamp 100 is shown to be about perpendicular (i.e., 90 degrees) in FIGS. 2A-2B, this rotational orientation can be at many different angles. For example, a first fixed rotational position of surgical fiducial marker positioner 200 is shown to be slightly less than perpendicular or 90 degrees in FIG. 3A, and a second fixed rotational position is shown to be about 45 degrees in FIG. 3B. Such fixed rotational positions can extend between 0 and 180 degrees and even beyond in various arrangements. A discrete number of fixed rotational positions can be achieved in some arrangements by way of mating features of positioner coupling component 250 and clamp coupling component 150, as set forth in greater detail below.

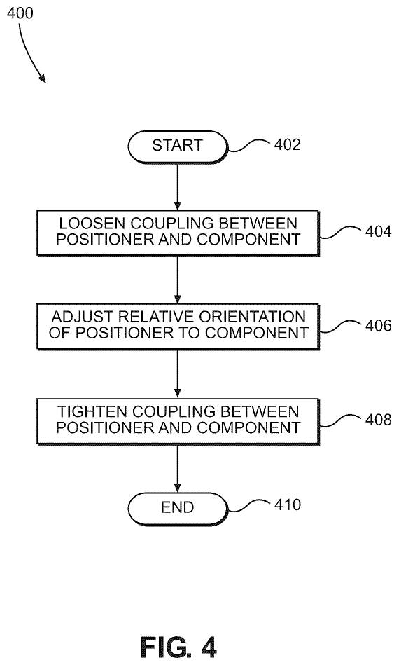

Moving next to FIG. 4, a flowchart of an example summary method of using a surgical fiducial marker positioner is provided. Summary method 400 can represent one broad aspect of various overall methods of use for a surgical fiducial marker positioner, and it will be understood that other steps, features, and details of such a broad aspect and overall methods of use are not provided here for purposes of simplicity. After a start step 402, a first process step 404 can involve loosening a coupling between the surgical fiducial marker positioner and a separate surgical component, which can be a spinous process clamp as one possible example. This can involve loosening a thumbscrew, wing nut, or other coupling arrangement between a positioner coupling component and a corresponding coupling component of the clamp or other separate surgical component, for example. Also, this can take place when the coupling arrangement is already tightened with the surgical fiducial marker positioner being at a first fixed position relative to the separate surgical component. Step 404 can be manually or automatically performed, such as where a separate robotic system can be configured to adjust the positioning of the surgical fiducial marker positioner.

At a following process step 406, an orientation of the surgical fiducial marker positioner relative to the separate surgical component can be adjusted from a first fixed position to a second fixed position. This can involve moving the surgical fiducial marker positioner while the separate surgical component remains stationary, for example, such as by rotation about an axis through a positioner coupling component and a corresponding coupling component that remain loosely coupled, among other possible relative movements. Step 406 can be manually or automatically performed, such as where a separate robotic system can be configured to adjust the positioning of the surgical fiducial marker positioner.

The next process step 408 can involve tightening the coupling between the surgical fiducial marker positioner and the separate surgical component. This can be done when the surgical fiducial marker positioner is at the second fixed position, for example, and the tightened coupling can prevent further relative movements between the surgical fiducial marker positioner and the separate surgical component. Step 408 can be manually or automatically performed, such as where a separate robotic system can be configured to adjust the positioning of the surgical fiducial marker positioner. The method can then end at end step 410.

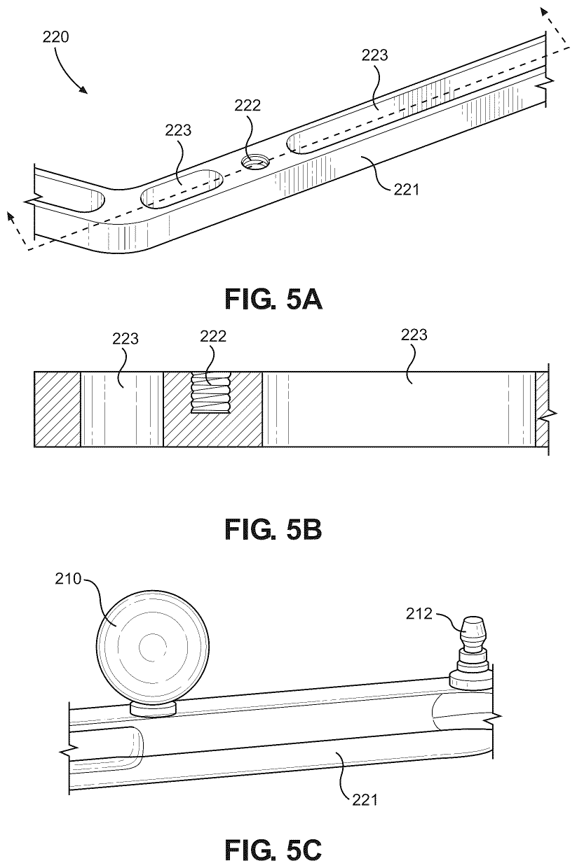

Transitioning now to FIGS. 5A and 5B, an example arm segment of a surgical fiducial marker positioner is shown in top perspective and side cross-section views respectively. As noted above, a surgical fiducial marker positioner can include a rigid frame 220 that includes multiple arm segments 221 arranged into a polygon shape, such as by forming or otherwise coupling all of the arm segments end to end to form the frame. Again, each arm segment 221 can have one or more marker openings 222 and one or more structural openings 223 extending into its material from a top surface thereof. Some or all of these marker openings 222 and structural openings 223 can extend all the way through arm segment 221.

FIG. 5C illustrates in side perspective view an example alternative surgical fiducial marker positioner arm segment with a fiducial marker and fiducial marker coupler coupled thereto. This arm segment 221 can include multiple fiducial marker couplers 212 that insert into marker openings along a top surface of the arm segment such that surgical fiducial markers 210 can be fitted onto the fiducial marker couplers, such as by way of holes or openings in the surgical fiducial markers. In some arrangements, all of these fittings or couplings between arm segment 221 and fiducial marker couplers 212, and also the fiducial marker couplers and surgical fiducial markers 210 can be removable such that the fiducial markers can be removed and reinstalled or replaced, as will be readily understood by those of skill in the art.

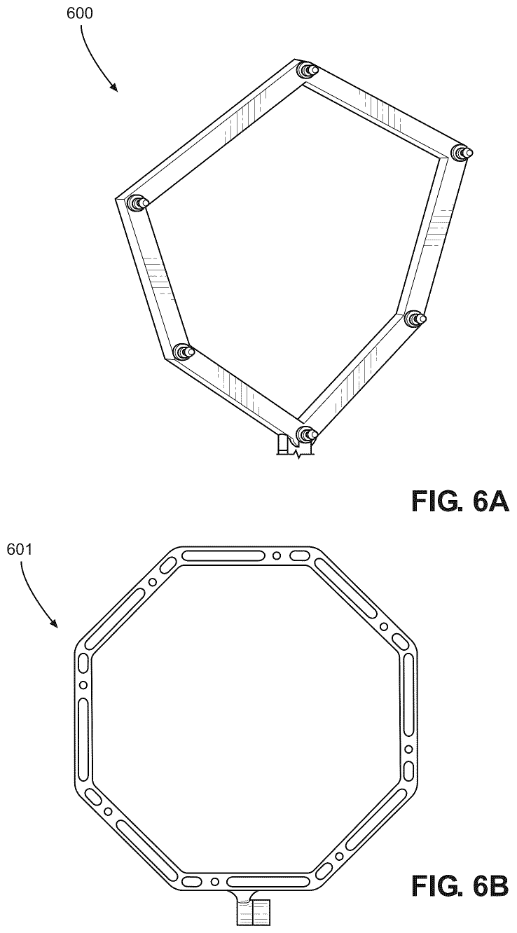

Moving next to FIGS. 6A and 6B, various alternative surgical fiducial marker positioners are depicted in top plan view. Surgical fiducial marker positioner 600 can have an alternative frame arrangement arranged along a plane and having an irregular hexagonal shape, for example. Also, fiducial marker couplers can be located at joints or intersections between arm segments as shown rather than along the arm segments. Although six surgical fiducial markers and six arm segments arranged into a hexagon shape have been shown for purposes of illustration for surgical fiducial marker positioners 200 and 600, it will be understood that a suitable surgical fiducial marker positioner may also have more or fewer fiducial markers arranged along more or fewer arm segments arranged into other shapes. Such other shapes can include rectangles, pentagons, octagons, for example, among other possible shapes. As another non-limiting illustrative example, surgical fiducial marker positioner 601 can have a rigid frame arranged along a plane into an octagon shape with eight fixed locations for fiducial marker couplers and fiducial markers. Other arrangements can include frames having seven arm segments arranged into an irregular shape, and other shapes and amounts of arm segments are also possible, as will be readily appreciated.

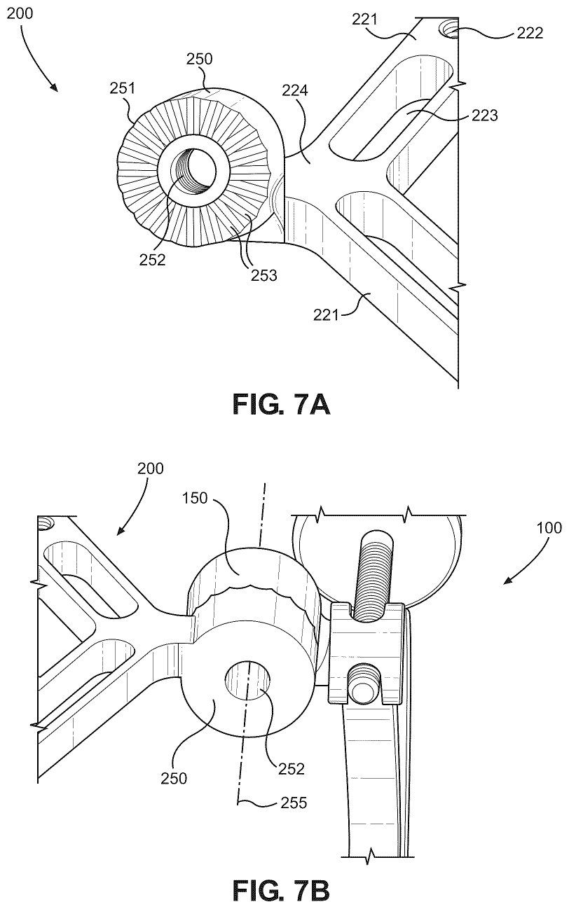

Continuing with FIGS. 7A and 7B, an example positioner coupling component for a surgical fiducial marker positioner is illustrated in perspective views both alone and also as being rotationally coupled to a clamp coupling component for a spinous process clamp. Again, surgical fiducial marker positioner 200 can include multiple arm segments 221 having marker openings 222 and optional structural openings 223. A neck 224 can extend from one of arm segments 221 or a junction between arm segments for attaching or coupling positioner coupling component 250. In some arrangements, neck 224 and positioner coupling component 250 can be integrally formed with the rigid frame having arm segments 221.

Positioner coupling component 250 can define a cylindrical shape having a vertically oriented circular mating face 251 and a threaded central opening 252 extending at least partially therethrough. Mating face 251 can have a plurality of ridges 253 that can be arranged in an axial pattern for mating with a similar axial pattern of ridges on a mating face of clamp coupling component 150. In addition to having a corresponding mating face that is configured to mate and operate with mating face 251, clamp coupling component 150 can similarly have a threaded central opening arranged to align with the threaded central opening 252 of positioner coupling component 250 when both mating faces are engaged.

In various arrangements, clamp coupling component 150 can be affixed to and can be stationary with spinous process clamp 100, while positioner coupling component 250 can be affixed to surgical fiducial marker positioner 200 and configured to rotate the entire surgical fiducial marker positioner with respect to the clamp coupling component. Rotation can be about a horizontal axis 255 that extends through the centers of threaded central opening 252 and the corresponding threaded central opening of clamp coupling component 150, such that the position of entire surgical fiducial marker positioner 200 is rotated about this axis. As will be readily appreciated, rotation of positioner coupling component 250 with respect to stationary clamp coupling component 150 can result in the relative rotation of its mating face 251 with a corresponding mating face of the clamp coupling component.

Ridges 253 on mating face 251 can fit snugly between corresponding ridges on the mating face of clamp coupling component 150, such that multiple discrete rotational orientations can be achieved. For example, where positioner coupling component 250 has a mating face 251 with ten axially arranged ridges 253, then clamp coupling component 150 can have a matching mating face with ten similarly axially arranged ridges, and this can facilitate at least ten different discrete rotational positions of surgical fiducial marker positioner 200 with respect to the spinous process clamp 100. Of course, more or fewer axially arranged ridges are also possible, and positioner coupling component 250 as shown can have twenty such axially arranged ridges 253 for twenty different discrete rotational positions in this particular illustrative example. In some embodiments, a thumbscrew, wing nut, or other coupling arrangement (not shown) can be used within threaded internal opening 252 and corresponding threaded internal opening of clamp coupling component 150 to loosen and tighten positioner coupling component 250 with respect to clamp coupling component 150. As will be readily appreciated, loosening such a coupling arrangement can allow for the ready rotational adjustment of surgical fiducial marker positioner 200 with respect to spinous process clamp 100, while tightening such a coupling arrangement can result in affixing or setting a discrete rotational position of the surgical fiducial marker positioner with respect to the spinous process clamp or other separate surgical component. One possible coupling arrangement between positioner coupling component 250 and clamp coupling component 150 can involve a thumbscrew and threaded post that can insert into threaded internal opening 252, details of which are shown in related U.S. patent application Ser. No. ______ entitled “SPINOUS PROCESS CLAMP,” which application is again hereby incorporated by reference in its entirety. Of course, other suitable coupling arrangements are also possible.

While clamp coupling component 150 and positioner coupling component 250 are shown as being vertically oriented with respect to spinous process clamp 100 in FIGS. 7A-B and other illustrative examples herein, it will be readily appreciated that these coupling components can be oriented horizontally or otherwise, such that relative rotation can be about a differently oriented axis. It is also contemplated that more than one rotational axis can be used for adjusting the positioning of surgical fiducial marker positioner 200 in some embodiments. For example, a secondary mating coupling component arrangement can allow for rotation of the surgical fiducial marker positioner 200 with respect to the spinous process clamp 100 about a vertical axis as well as a horizontal axis.

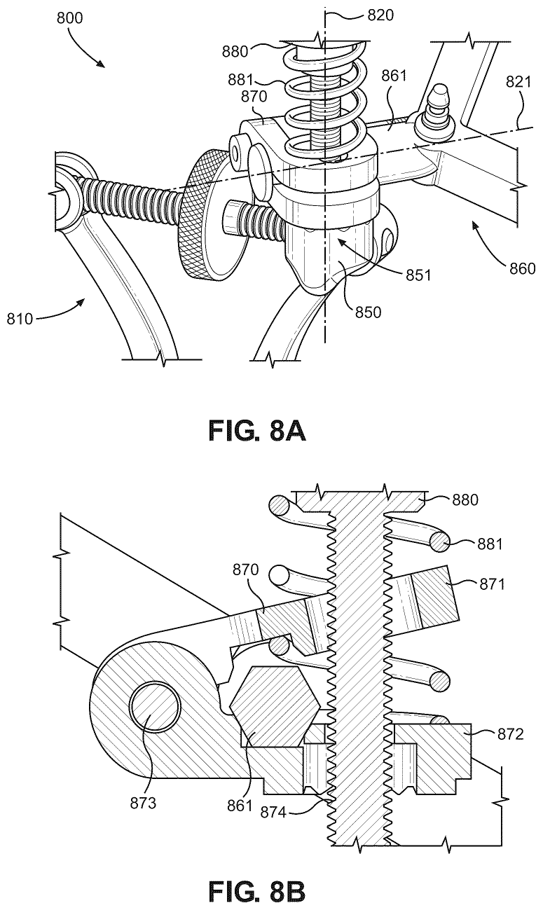

FIGS. 8A and 8B illustrate an example alternative coupling arrangement between a surgical fiducial marker positioner and spinous process clamp in perspective and cross-section views respectively. Alternative surgical fiducial marker system 800 can generally include spinous process clamp 810 and surgical fiducial marker positioner 860, both of which can be substantially similar in many respects and details to spinous process clamp 100 and surgical fiducial marker positioner 200 above. Unlike surgical fiducial marker system 10 disclosed in detail above, however, alternative surgical fiducial marker system 800 can have a coupling arrangement that facilitates relative rotation about multiple axes, such as vertical axis 820 and horizontal axis 821. Spinous process clamp 810 can have an alternative clamp coupling component 850 while surgical fiducial marker positioner 860 can have an alternative positioner coupling component 870 or arrangement to facilitate this alternative coupling arrangement.

As shown, clamp coupling component 850 can be coupled to or located proximate a top distal end of an arm of spinous process clamp 810, while positioner coupling component 870 can be rotationally coupled around a neck 861 extending from surgical marker positioner 860. A mating face 851 at a top surface of clamp coupling component 850 can have axial ridges and be configured to mate with a similar mating face 874 along a bottom surface of positioner coupling component 870. Such a rotational mating arrangement can be similar to the rotational mating arrangement set forth above between clamp coupling component 150 and positioner coupling component 250, except with mating faces 851, 874 facilitating a horizontal rotation of fiducial marker positioner 860 relative to stationary spinous process clamp 810 about vertical axis 820.

To facilitate an axial rotation of fiducial marker positioner 860 relative to stationary spinous process clamp 810 about horizontal axis 821, a portion of neck 861 can extend into and can be configured to rotate within positioner coupling component 870. This can be facilitated by positioner coupling component 870 having a top portion 871 and a bottom portion 872 that are hinged or coupled about a pivot pin 873 such these top and bottom portions can clamp onto and release off from neck 861. As shown, at least a portion of neck 861 inside of positioner coupling component 870 can have a discrete polygonal cross-section shape to facilitate discrete rotational amounts and positions of the neck. Thumbscrew 880 and bias spring 881 can facilitate the ability to loosen and tighten the fit of positioner coupling component 870 around neck 861, as well as to loosen and tighten the fit of mating faces 851 and 874, as will be readily appreciated by those of skill in the art. Although this alternative coupling arrangement of alternative surgical fiducial marker system 800 having multiple axes of relative rotation has been provided for purposes of illustration, it will be understood that other types of coupling arrangements between a surgical fiducial marker positioner and a clamp or other separate surgical component can also be used, and that such other types of coupling arrangements can have more or few axes or rotation, other ways of loosening and tightening coupling components, and even other forms of relative movement or positioning besides rotational movement.

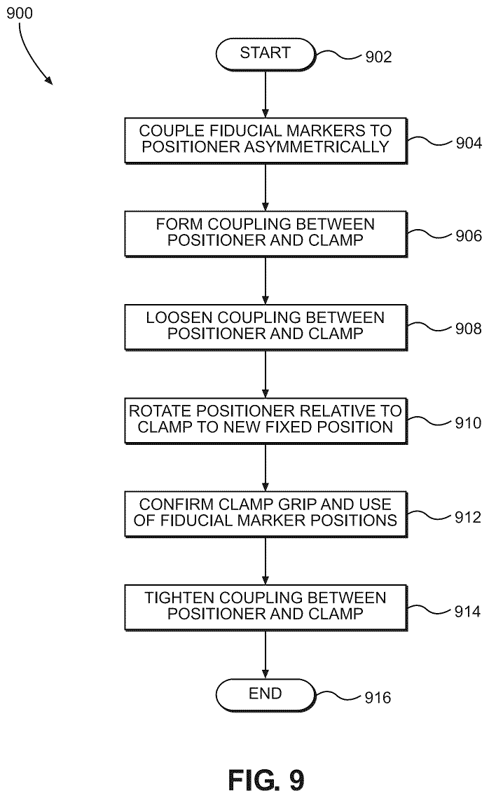

Next, FIG. 9 provides a flowchart of an example detailed method 900 of using a surgical fiducial marker positioner. Detailed method 900 can represent one possible way of using a surgical fiducial marker positioner, and it will be understood that various other steps, features, and details of such a detailed method are not provided here for purposes of simplicity. After a start step 902, a first process step 904 can involve coupling surgical fiducial markers to a surgical fiducial marker positioner. This can involve removably coupling the surgical fiducial markers to the surgical fiducial marker positioner at fixed positions relative to each other to form an asymmetrical fixed positional arrangement of surgical fiducial markers, and can also include the use of fiducial marker couplers that removably couple to both the fiducial markers and the surgical fiducial marker positioner. Step 904 can be manually or automatically performed, such as where a separate robotic system can be configured to couple the surgical fiducial markers to the surgical fiducial marker positioner.

At the next process step 906, a coupling can be formed between the surgical fiducial marker positioner and a separate surgical component, such as a spinous process clamp. This can be a rotational coupling involving threaded openings in mating components, a threaded bar, and one or more thumbscrews, wing nuts, or other suitable tighteners, for example. Step 906 can be manually or automatically performed, such as where a separate robotic system can be configured to couple a surgical fiducial marker positioner to a spinous process clamp or another separate surgical component.

A following process step 908 can involve loosening the coupling between the surgical fiducial marker positioner and spinous process clamp or other separate surgical component. This can involve loosening a thumbscrew, wing nut, or other coupling arrangement between a positioner coupling component and a corresponding coupling component of the clamp or other separate surgical component. This can take place when the coupling arrangement is already tightened with the surgical fiducial marker positioner being at a first fixed position relative to the separate surgical component, or alternatively this step can be skipped if the formed coupling is already loose such that rotational adjustments are possible. Step 908 can be manually or automatically performed, such as where a separate robotic system can be configured to adjust the positioning of the surgical fiducial marker positioner.

At a subsequent process step 910, the surgical fiducial marker positioner can be rotated relative to the clamp or other separate surgical component to a new fixed position. This can involve rotating the surgical fiducial marker positioner while the clamp or other separate surgical component remains stationary, such as by rotation about an axis through a positioner coupling component and a corresponding coupling component that remain loosely coupled. Rotation can be from a first fixed position to a second fixed position, for example. Step 910 can be manually or automatically performed, such as where a separate robotic system can be configured to adjust the positioning of the surgical fiducial marker positioner.

The next process step 912 can involve confirming that the spinous process clamp firmly grips a spinous process of a patient and also that the asymmetrical fixed positional arrangement of surgical fiducial markers is usable by a separate surgical system when the surgical fiducial marker positioner is at the second rotational fixed position. Step 912 can be manually or automatically performed, such as where a separate robotic system can be configured to register the visibility and positions of all surgical fiducial markers and that none of these fiducial markers are moving or otherwise unstable when the overall system is set.

At the following process step 914, the coupling can be tightened between the surgical fiducial marker positioner and the clamp or other separate surgical component. This can be done when the surgical fiducial marker positioner is at the second fixed position, for example, and the tightened coupling can prevent further relative movements between the surgical fiducial marker positioner and the clamp or other separate surgical component. Step 914 can be manually or automatically performed, such as where a separate robotic system can be configured to adjust the positioning of the surgical fiducial marker positioner. The method can then end at end step 916.

For foregoing method 900, it will be appreciated that not all process steps are necessary, and that other process steps may be added in some arrangements. For example, removing old fiducial markers and cleaning or sterilizing the surgical fiducial marker positioner might take place before coupling new fiducial markers may take place in some arrangements. Other steps may involve functions of the associated spinous process clamp or other separate surgical component. Furthermore, the order of steps may be altered in some cases, and some steps may be performed simultaneously. For example, step 912 may be performed after step 914 in some cases. Although known process steps are provided for the various techniques in method 900, it will be appreciated that any other suitable similar method for using a surgical fiducial marker positioner can also be used. Other variations and extrapolations of the disclosed methods will also be readily appreciated by those of skill in the art.

### Magnetically Couplable Fiducial Marker Arrays

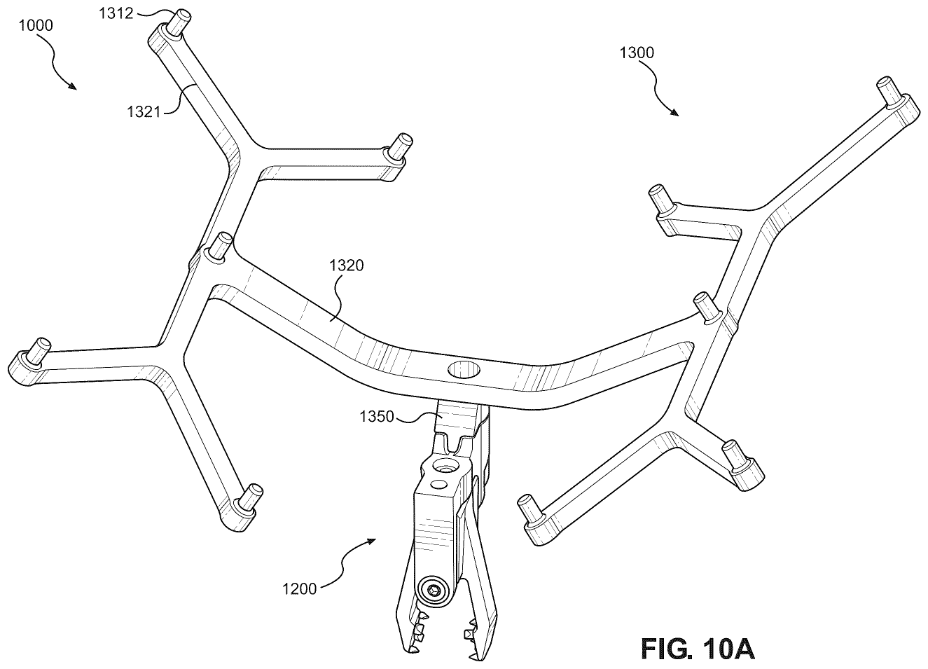

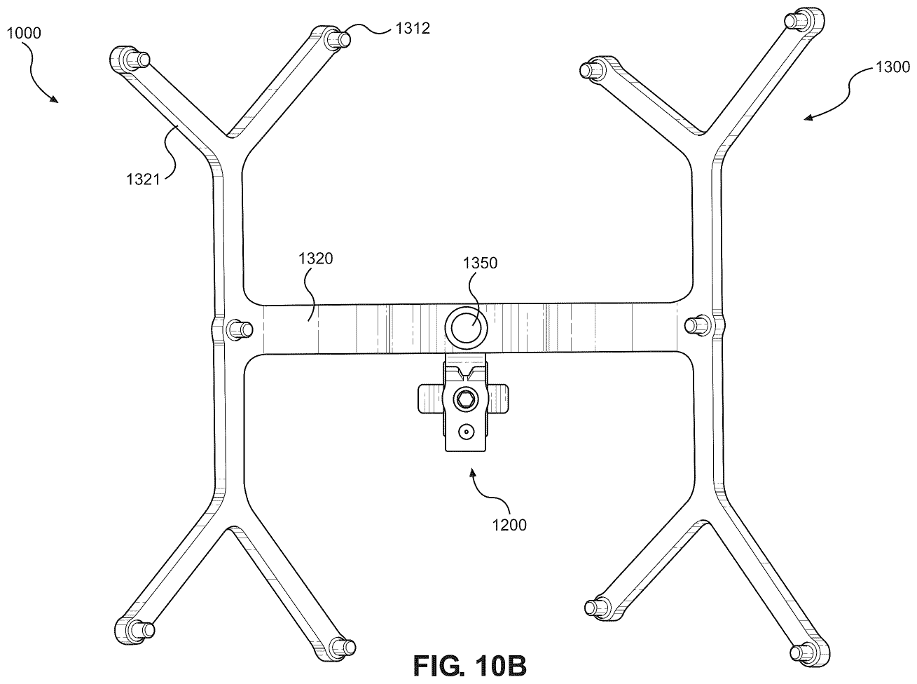

Transitioning now to FIGS. 10A and 10B, an example alternative surgical fiducial marker system having a surgical spinous process clamp and a magnetically couplable fiducial marker array is illustrated in front perspective and top plan views respectively. Alternative surgical fiducial marker system 1000 can generally include a surgical spinous process clamp 1200 and a magnetically couplable fiducial marker array 1300. It will be understood that surgical spinous process clamp 1200 can be different in various ways from spinous process clamp 100 above while still performing some of the same functions. Similar to spinous process clamp 100 above (which can also be considered surgical in nature), for example, surgical spinous process clamp 1200 can be used to clamp to one of various spinous processes along the spine of a patient, such as during a surgery. Various aspects of surgical spinous process clamp 1200 relevant to how this clamp couples to magnetically couplable fiducial marker array 1300 are set forth in detail below. Other aspects of surgical spinous process clamp 1200 are set forth in greater detail in related U.S. Provisional Patent Application No. ______, titled “SURGICAL SPINOUS PROCESS CLAMP,” which application is again hereby incorporated by reference in its entirety herein.

It will also be understood that magnetically couplable fiducial marker array 1300 can be different in various ways from surgical fiducial marker positioner 200 above while still performing some of the same functions. Similar to surgical fiducial marker positioner 200 above (which can also be considered a fiducial marker array), magnetically couplable fiducial marker array 1300 can be used to position surgical fiducial markers in space while coupled to surgical spinous process clamp 1200. These surgical fiducial markers (not shown) can be identical or substantially similar to surgical fiducial markers 210 above and can be coupled to magnetically couplable fiducial marker array 1300 by way of fiducial marker couplers 1312 located at each position where a surgical fiducial marker should be coupled.

Magnetically couplable fiducial marker array 1300 can include a rigid body 1320 configured to support a plurality of surgical fiducial markers (not shown) at fixed positions relative to each other along the rigid body by way of fiducial marker couplers 1312 to form a fixed positional arrangement of surgical fiducial markers that can be asymmetrical, as shown. Magnetically couplable fiducial marker array 1300 can be a full array having surgical fiducial markers at rigid body branches 1321 on both sides of magnetic coupling arrangement 1350 that couples the full array to surgical spinous process clamp 1200. Each side or half of magnetically couplable fiducial marker array 1300 can include a support portion of the rigid body 1320 that can be arranged along a surgical fiducial marker support plane, such that all surgical fiducial markers on each side are aligned along the same plane. The fixed positional arrangement of all surgical fiducial markers (located at fiducial marker couplers 1312 when installed) can be asymmetrical. In some arrangements, each half or side of magnetically couplable fiducial marker array 1300 can also include its own fixed positional arrangement of its half or side of the surgical fiducial markers that can also by asymmetrical.

As shown, magnetically couplable fiducial marker array 1300 can include coupling arrangement 1350 integrally formed with, affixed to, or otherwise coupled to rigid body 1320. Coupling arrangement 1350 configured to couple the fiducial marker array 1300 to a separate surgical device (e.g., surgical spinous process clamp 1200) at a first fixed position relative to the separate surgical device.

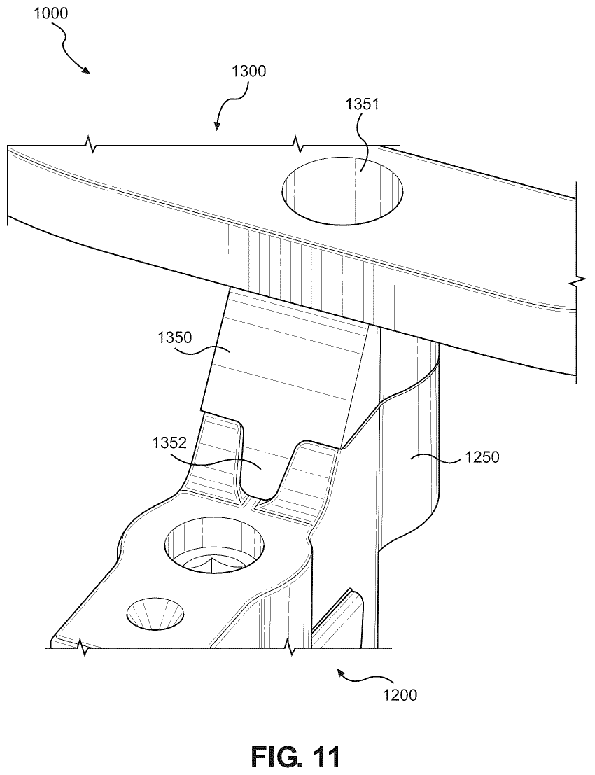

Moving next to FIG. 11, an example magnetic coupling between a surgical spinous process clamp and a magnetically couplable fiducial marker array is illustrated in front perspective view. Again, alternative surgical fiducial marker system 1000 can generally include a surgical spinous process clamp 1200 and a magnetically couplable fiducial marker array 1300, and these components can be magnetically coupled together. Surgical spinous process clamp 1200 can include clamp magnetic coupling arrangement 1250 including one or more magnetic components, while magnetically couplable fiducial marker array 1300 can include array magnetic coupling arrangement 1350 that includes one or more array magnetic components configured to form a magnetic coupling to the clamp magnetic components. A cavity 1351 within magnetic coupling arrangement 1350 can be arranged to hold one or more magnetic components therein. Such magnetic components can be actual magnets or can be metallic items that are configured to interact with magnets, as will be readily appreciated. One or more geometric features 1352 on array magnetic coupling arrangement 1350 can be sized and shaped so as to fit in a particular way with mating geometric features on clamp magnetic coupling arrangement 1250.

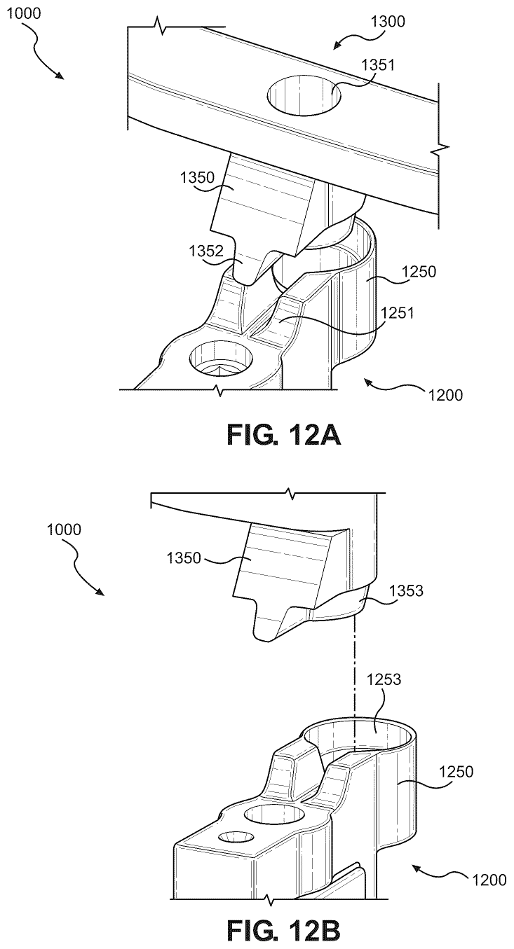

FIG. 12A illustrates in front perspective view the magnetic coupling of FIG. 11 being undone by removing the magnetically couplable fiducial marker array from the surgical spinous process clamp, while FIG. 12B illustrates in front perspective view the magnetically couplable fiducial marker array being completely removed from the surgical spinous process clamp of FIG. 12A. As shown in FIGS. 12A and 12B, array geometric features 1352 on array magnetic coupling arrangement 1350 can mate in a specific way with clamp geometric features 1251 on clamp magnetic coupling arrangement 1250. Protrusion 1353 at the bottom of magnetic coupling arrangement 1350 can fit with a corresponding recess within clamp magnetic coupling arrangement 1250 as shown, such that the magnetic items in each component are placed in close proximity to each other for a sufficient magnetic attraction to take place to hold the entire magnetically couplable fiducial marker array 1300 as magnetically coupled against surgical spinous process clamp 1200.

It will be readily appreciated that the physical coupling between surgical spinous process clamp 1200 and magnetically couplable fiducial marker array 1300 can be done in only one fixed orientation due to the array geometric features 1352 fitting only one way with the clamp geometric features 1251. Accordingly, if the magnetic coupling between the fiducial marker array and the clamp is ever manually disturbed or removed, then the coupling can also be reformed and any calibration previously done with the fiducial markers will still be good.

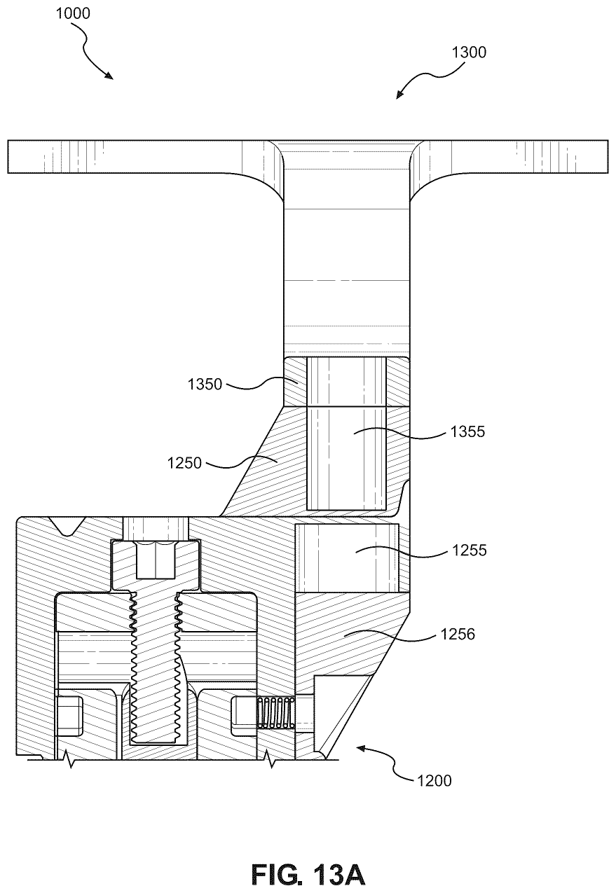

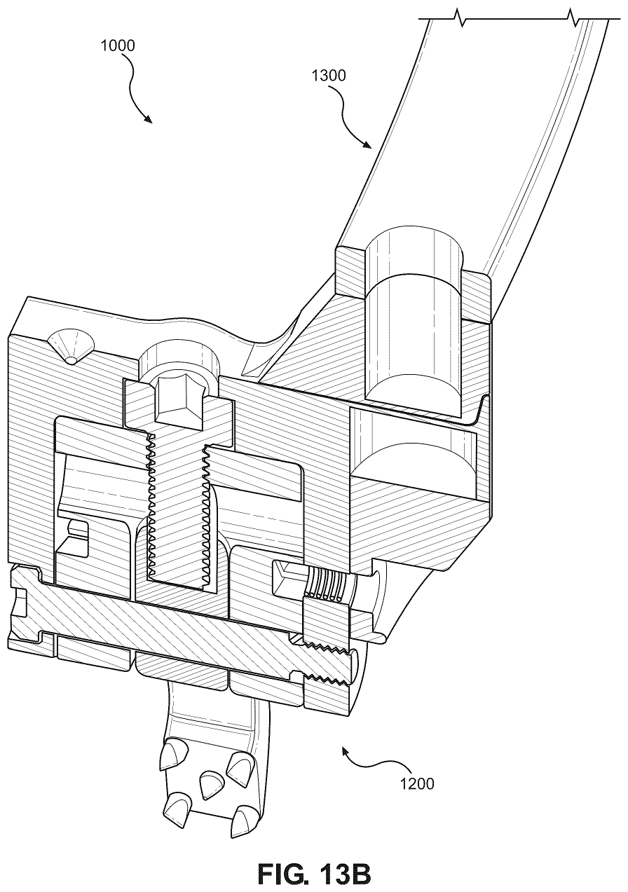

FIGS. 13A and 13B illustrate in side cross-section and perspective cross-section views the magnetic coupling between surgical spinous process clamp 1200 and magnetically couplable fiducial marker array 1300, as similarly arranged in FIG. 11. These views provide added perspectives of the various components and features illustrated above. In addition, an array magnet cavity 1355 and clamp magnet cavity 1255 can be seen. Cavities 1255, 1355 can be arranged to hold one or more magnetic components each. Cover 1256 can be coupled to a main body of surgical spinous process clamp 1200 to hold the clamp magnetic components in clamp magnet cavity 1255, while a separate cover or lid (not shown) can function to hold the array magnetic components in array magnet cavity 1355.

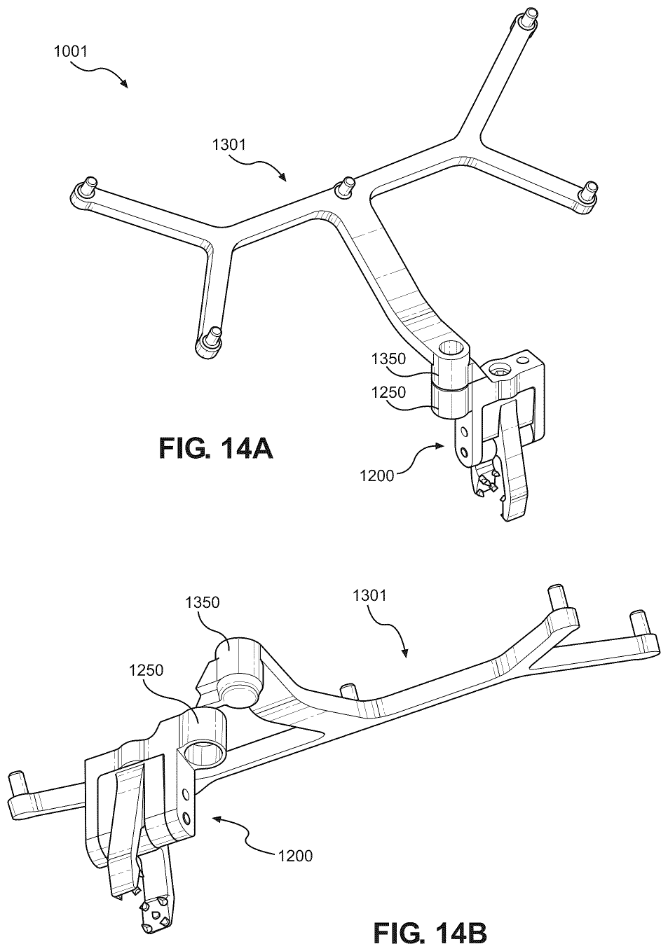

FIGS. 14A and 14B illustrates in top perspective and bottom perspective views another example alternative surgical fiducial marker system having a surgical spinous process clamp and a magnetically couplable half fiducial marker array. Alternative surgical fiducial marker system 1001 can be substantially similar to system 1000, except that the fiducial marker array is only half of an array to one side of the clamp 1200, as shown. As such, alternative surgical fiducial marker system 1001 generally includes a surgical spinous process clamp 1200 and a magnetically couplable half fiducial marker array 1301. In this system 1001, all components can be identical or similar, except for the missing half of a fiducial marker array. This arrangement can be advantageous to allow a surgeon or operator to be able to more readily work on a spinous process where the half array is missing, while still having enough fiducial markers on the remaining half array to be able to facilitate system and item tracking in augmented reality environment.

In various embodiments, an overall surgical fiducial marker system can include a surgical spinous process clamp 1200 and three different magnetically couplable half fiducial marker arrays. These arrays can include the full array 1300 previously shown, the half array 1301 shown in FIGS. 14A and 14B, and another half array (not shown) that is a mirror to array 1301. Each of these three different magnetically couplable half fiducial marker arrays can be interchangeably used with the same surgical spinous process clamp 1200 and all can couple to the clamp coupling component 1250 in the same manner with the same type of array coupling component 1350. In some arrangements, the locations of the surgical fiducial markers on all three of the arrays can be identical, such that removing one array and replacing with another can result in calibration of fiducial marker locations not being needed for the newly installed array when calibration had been done on the previous array.

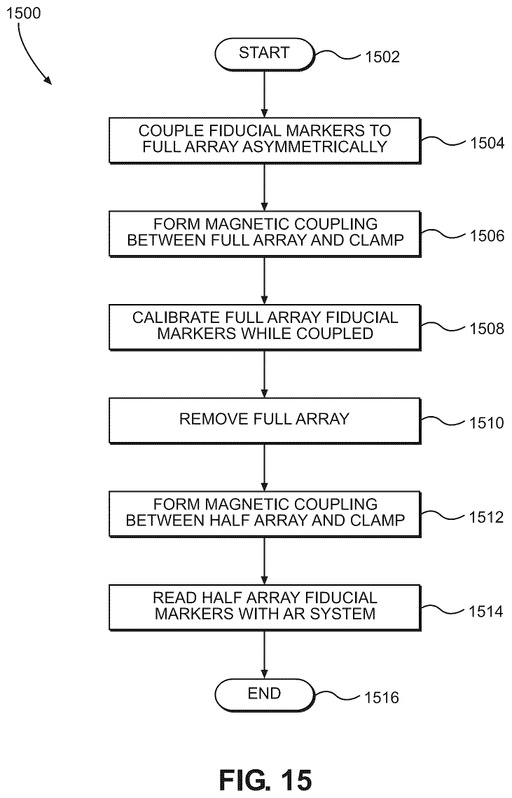

FIG. 15 illustrates a flowchart of an example method of using a magnetically couplable fiducial marker array. Method 1500 can represent one possible way of using a magnetically couplable fiducial marker array, and it will be understood that various other steps, features, and details of such a detailed method are not provided here for purposes of simplicity. After a start step 1502, a first process step 1504 can involve coupling surgical fiducial markers to a magnetically couplable full fiducial marker array. This can involve removably coupling the surgical fiducial markers to the magnetically couplable full fiducial marker array at fixed positions relative to each other to form an asymmetrical fixed positional arrangement of surgical fiducial markers, and can also include the use of fiducial marker couplers that removably couple to both the fiducial markers and the magnetically couplable fiducial marker array. Step 1504 can be manually or automatically performed, such as where a separate robotic system can be configured to couple the fiducial markers to the magnetically couplable fiducial marker array.

At the next process step 1506, a magnetic coupling can be formed between the magnetically couplable full fiducial marker array and a separate surgical device, such as a spinous process clamp. This can be a magnetic coupling such as that which is illustrated and described above and can include magnets or magnetic components in both the fiducial marker array and the separate spinous process clamp or other medical device, and can also involve specific mating geometries in the devices such that only one orientation is possible. Step 1506 can be manually or automatically performed, such as where a separate robotic system can be configured to couple a magnetically couplable fiducial marker array to a spinous process clamp or another separate surgical component.

A following process step 1508 can involve calibrating the locations in space of some or all of the surgical fiducial markers on the magnetically couplable full fiducial marker array. Step 1508 can be manually or automatically performed, such as where a separate augmented reality calibration system can be programmed to calibrate surgical fiducial markers automatically using system software, cameras, detectors, and other equipment.

At a subsequent process step 1510, the magnetically couplable full fiducial marker array can be removed from the spinous process clamp or other separate device. Step 1510 can be manually or automatically performed, such as where a separate robotic system can be configured to handle and remove the full fiducial marker array.

The next process step 1512 can involve forming a magnetic coupling between a magnetically couplable half fiducial marker array and the spinous process clamp. This can involve installing a half marker array such as that which is shown in FIGS. 14A and 14B above. Step 1512 can be manually or automatically performed, such as where a separate robotic system can be configured to handle and install the half fiducial marker array.

At the following process step 1514, the locations of the surgical fiducial markers on the magnetically couplable half fiducial marker array can be read by an augmented reality system. Step 1514 can be automatically performed, such as where a separate augmented reality calibration system can be programmed to detect or read surgical fiducial marker locations automatically using system software, cameras, detectors, and other equipment. The method can then end at end step 916.

For foregoing method 900, it will be appreciated that not all process steps are necessary, and that other process steps may be added in some arrangements. For example, removing old fiducial markers and cleaning or sterilizing the surgical fiducial marker positioner might take place before coupling new fiducial markers may take place in some arrangements. Other steps may involve functions of the associated spinous process clamp or other separate surgical component. Furthermore, the order of steps may be altered in some cases, and some steps may be performed simultaneously. For example, step 912 may be performed after step 914 in some cases. Although known process steps are provided for the various techniques in method 900, it will be appreciated that any other suitable similar method for using a surgical fiducial marker positioner can also be used. Other variations and extrapolations of the disclosed methods will also be readily appreciated by those of skill in the art.

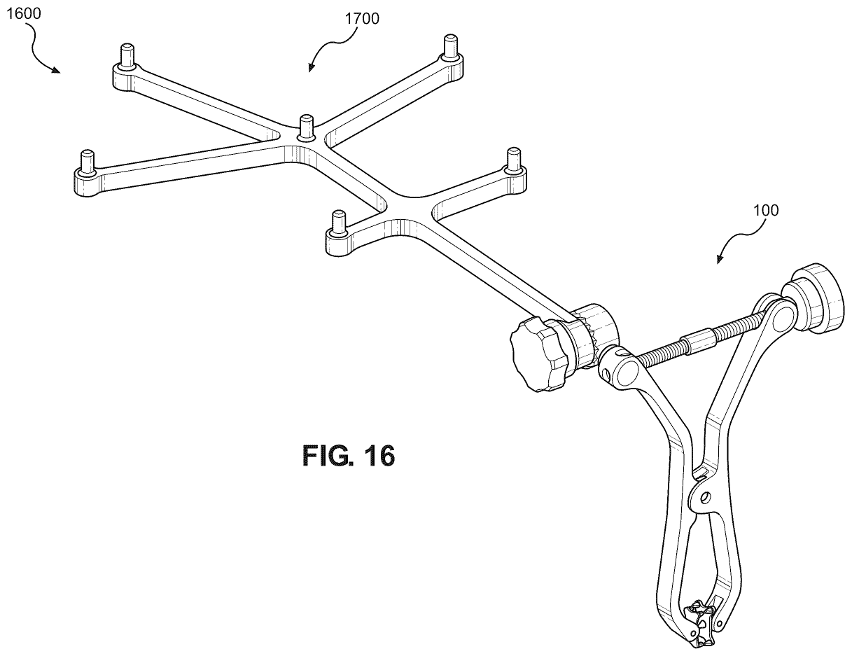

FIG. 16 illustrates in top perspective view yet another alternative surgical fiducial marker system with the spinous process clamp of FIG. 2A and an alternative surgical fiducial marker array. Alternative surgical fiducial marker system provides another example of the different types of fiducial marker arrays that can be used with any type of spinous process clamp or other medical device or item. For example, spinous process clamp 100 as disclosed above can be rotatably coupled to alternative surgical fiducial marker array 1700, which can have multiple branches rather than a hexagonal shape. Other arrangements and types of fiducial marker arrays are also possible, as will be readily appreciated.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims.

What is claimed is:

1. A fiducial marker array, comprising:
a rigid body configured to support a plurality of surgical fiducial markers at fixed positions relative to each other along the rigid body to form a fixed positional arrangement of surgical fiducial markers, wherein at least a support portion of the rigid body is arranged along a surgical fiducial marker support plane and the fixed positional arrangement of surgical fiducial markers is asymmetrical; and
a coupling arrangement coupled to the rigid body, wherein the coupling arrangement is configured to couple the fiducial marker array to a separate surgical device at a first fixed position relative to the separate surgical device.

2. The fiducial marker array of claim 1, further comprising:
the plurality of surgical fiducial markers.

3. The fiducial marker array of claim 1, wherein the separate surgical device is a surgical spinous process clamp.

4. The fiducial marker array of claim 1, wherein the coupling arrangement includes one or more array magnetic components configured to form a magnetic coupling to one or more separate magnetic components of the separate surgical device.

5. The fiducial marker array of claim 4, wherein the magnetic coupling results in the fixed positional arrangement of surgical fiducial markers being set at a constant orientation and location with respect to the separate surgical device while the fiducial marker array remains magnetically coupled to the separate surgical device.

6. The fiducial marker array of claim 5, wherein the constant orientation and location is repeatable when the fiducial marker array is uncoupled from and recoupled to the separate surgical device such that recalibration of the fiducial marker array is unnecessary.

7. The fiducial marker array of claim 1, wherein the support portion of the rigid body is configured to support all of the plurality of surgical fiducial markers.

8. The fiducial marker array of claim 1, wherein the support portion of the rigid body includes a primary branch and multiple secondary branches extending from the primary branch at different locations, each secondary branch including at least one location configured to support one of the plurality of surgical fiducial markers.

9. The fiducial marker array of claim 8, wherein the multiple secondary branches include at least four secondary branches.

10. The fiducial marker array of claim 1, wherein the rigid body extends laterally in only one direction away from the coupling arrangement.

11. The fiducial marker array of claim 1, wherein the rigid body extends laterally in exactly two opposite directions away from the coupling arrangement.

12. A magnetically couplable fiducial marker array, comprising:
a rigid body configured to support a plurality of surgical fiducial markers at fixed positions relative to each other along the rigid body to form a fixed positional arrangement of surgical fiducial markers, wherein at least a support portion of the rigid body is arranged along a surgical fiducial marker support plane and the fixed positional arrangement of surgical fiducial markers is asymmetrical;
the plurality of surgical fiducial markers; and
a magnetic coupling arrangement formed at the rigid body and having one or more array magnetic components, wherein the magnetic coupling arrangement is configured to form a magnetic coupling between the one or more array magnetic components and one or more separate magnetic components of a separate surgical spinous process clamp at a first fixed position relative to the separate surgical spinous process clamp.

13. A method of using a magnetically couplable fiducial marker array, comprising:
removably coupling a plurality of surgical fiducial markers to a magnetically couplable full fiducial marker array at fixed positions relative to each other to form an asymmetrical fixed positional arrangement of surgical fiducial markers;
forming a magnetic coupling between the magnetically couplable full fiducial marker array and a separate surgical component;
calibrating the locations of the plurality of surgical fiducial markers while the magnetically couplable full fiducial marker array is magnetically coupled to the separate surgical component;
removing the magnetically couplable full fiducial marker array from the separate surgical component;
forming a magnetic coupling between a magnetically couplable half fiducial marker array and the separate surgical component; and
reading automatically surgical fiducial markers on the magnetically couplable half fiducial marker array by an augmented reality system while the magnetically couplable half fiducial marker array is magnetically coupled to the separate surgical component. 14, The method of claim 13, wherein the separate surgical component is a surgical spinous process clamp.
